(19) United States
(12) Patent Application Publication
(10) Pub. No.: US 2004/0176830 A1
(43) Pub. Date: Sep. 9, 2004

(54) Epicardial electrode

(76) Inventor: H. Kenith Fang, Phoenix, AZ (US)

Correspondence Address: WILLIAM C. CAHILL, 155 PARK ONE, 2141 E. HIGHLAND AVENUE, PHOENIX, AZ, 85016, US

(21) Appl. No.: 10/383,087

(22) Filed: Mar. 6, 2003

(51) Int. Cl. A61N 1/05

(52) U.S. Cl. 607/129; 607/130

(57) ABSTRACT

An epicardial electrode (10) includes a generally parallelepiped flexible body (12). The epicardial electrode has an electrode element (22) attached to the center of a first side (14) for conveying electrical stimulation to cardiac muscle, and a lead (24) attached to the flexible body at a lead side (18). The lead has at least an insulated cathode conductor (26) electrically coupled to the electrode element. The epicardial electrode also has two pairs of prongs (31-34), electrically insulated from the electrode element, for anchoring the epicardial electrode to the heart. The tip (41-44) of each prong is dull. The flexible body has two elongate holes (51-52) on opposite sides of the flexible body sized to accept rods of a tool for flexing the epicardial electrode.

### BACKGROUND OF THE INVENTION

1. Field of the Invention

This invention relates to the field of electrical energy applicators that are placed in a human body at the heart, and in particular, to a patch or epicardial electrode employing an anchor other than a suture, for retaining the electrode in operative position on the surface of the heart.

2. Description of the Related Art

Prior art epicardial electrodes that are sutured into operative position on the heart are well known, as are their disadvantages. In order to facilitate suturing of the epicardial electrode, a thoracotomy is sometimes performed, which is disadvantageous. Alternatively, the heart can be accessed through much smaller incisions using thoracoscopic methods, but then suturing the epicardial electrode to the heart becomes very difficult. With either method of gaining access to the heart, suturing can cause unnecessary bleeding, especially if the epicardial electrode needs to be repositioned because of unsatisfactory lead position.

Epicardial electrodes that are anchored to the heart by means other than suturing are well known. For example, there are epicardial electrodes that use a helical element to anchor the epicardial electrode to the heart. In many prior art epicardial electrodes, the element that anchors the epicardial electrode to the heart also acts as the stimulating element. However, the disadvantages of using the element that anchors the epicardial electrode to the heart as the stimulating element, whether it is helical or of another shape, are also well known. Fibrosis can occur at the stimulating element itself. This is undesirable because fibrosis/scar formation has a higher electrical impedance than normal tissue, and can cause undesirable capture thresholds.

Accordingly, there have been numerous attempts to overcome the aforesaid disadvantages, such as by using separate elements for anchoring and for stimulating. Examples of prior art patents that disclose separate elements for anchoring and for stimulating include:

U.S. Pat. No. 4,066,085 entitled Contact Device for Muscle Stimulation, issued Jan. 3, 1978 to Hess, discloses a contact device having a plurality of fishhook-type barbs or needle-like members for attaching the contact device to the heart, and a separate, helical coil electrode to stimulate the muscle. The contact device of Hess does not address the disadvantages of fibrosis formation at the site of electrode implantation. In particular, Hess fails to disclose a dull attachment member.

U.S. Pat. No. 4,177,818 entitled Self Attachable Small-Toothed Electrode and a Forceps for Maneuvering It, issued Dec. 11, 1979 to De Pedro, discloses an electrode carrying member having four inwardly curved teeth, each tooth having a sharpened thin point, for embedding into the heart muscle, and a separate thin point constituting the myocardium stimulator. The electrode carrying member of De Pedro has the disadvantage of piercing the myocardium, which can lead to fibrosis/scar formation and its inherent problems with threshold capture. Specifically, De Pedro fails to disclose a dull tooth for embedding into the heart muscle.

U.S. Pat. No. 4,607,644 entitled Self-Suturing Porous Epicardial Electrode Assembly, issued Aug. 26, 1986 to Pohndorf, discloses an electrode assembly having two pairs of legs, each leg having a sharply pointed, outwardly projecting, curved prong for penetrating the myocardial wall and for embedding themselves firmly therein, to secure the electrode assembly to the myocardial wall, and a separate electrode contact for conducting heart-pacing electrical pulses to the heart muscle. However, Pohndorf fails to disclose a dull prong for embedding into the heart muscle as its anchoring mechanism.

Each of the known epicardial electrodes that uses separate elements for supplying electrical energy to the heart muscle and for anchoring the epicardial electrode to the heart disadvantageously have sharp, pointed, or needle-like anchoring elements. The sharp, pointed, or needle-like anchoring elements can cause lacerations to the myocardium, which cause unnecessary bleeding that results in formation of undesirable fibrosis or scarring.

Thus, what is needed is an epicardial electrode with separate elements for supplying electrical energy to the heart muscle and for anchoring the epicardial electrode to the heart, which has improved anchoring elements that is less traumatic to the underlying myocardium.

### SUMMARY OF THE INVENTION

Briefly described, and in accordance with a preferred embodiment thereof, the present invention relates to an epicardial electrode that includes a generally parallelepiped flexible body having a first side, a second side opposite the first side, a lead side, and a back side opposite the lead side. The epicardial electrode has an electrode element attached to the first side at the center of the first side for conveying electrical stimulation to cardiac muscle, and a lead attached to the flexible body at the lead side. The lead has at least an insulated cathode conductor electrically coupled to the electrode element. The epicardial electrode also has two pairs of prongs insulated from the electrode element, for anchoring the epicardial electrode to the heart. Each prong protrudes from the first side of the flexible body. The external tip of each prong is dull, thereby allowing anchoring of the epicardial electrode to the cardiac muscle with minimal trauma to the cardiac muscle.

The present invention also relates to an epicardial electrode that includes a generally parallelepiped flexible body having a first side, a second side opposite the first side, a lead side, and a back side opposite the lead side. The flexible body has two elongate holes, distal from the first side, on opposite sides of the flexible body. The elongate holes have an elongate axis parallel to the first side and perpendicular to the lead side. The epicardial electrode has an electrode element attached to the first side at the center of the first side for conveying electrical stimulation to cardiac muscle, and a lead attached to the flexible body at the lead side. The lead has at least an insulated cathode conductor electrically coupled to the electrode element. The epicardial electrode also has two pairs of prongs that protrude from the first side of the flexible body and that are insulated from the electrode element, which are for anchoring the epicardial electrode to the heart.

The present invention further relates to a method of placing the epicardial electrode in operative position on the heart, comprising the steps of: applying force to flex the flexible body such that the first side becomes convex; while flexed, positioning the epicardial electrode at a predetermined location on the heart such that the electrode element is in intimate contact with the heart; and, while the electrode element is in intimate contact with the heart, removing the force to allow the first side to return to a planar shape, causing the prongs to penetrate the myocardium, thereby anchoring the epicardial electrode to the heart.

### BRIEF DESCRIPTION OF THE DRAWINGS

The present invention will be described with greater specificity and clarity with reference to the following drawings, in which:

FIG. 1 is a perspective view of an epicardial electrode in accordance with the invention, showing two pairs of anchoring elements;

FIG. 2 is a front view of the epicardial electrode shown in a flexed position;

FIG. 3 is a front view of the epicardial electrode shown in a relaxed position;

FIG. 4 is a cross-sectional view of the epicardial electrode through cut-line 4-4 of FIG. 1;

FIG. 5 is a plan view of the epicardial electrode;

FIG. 6 is a cross-sectional view of the epicardial electrode through cut-line 6-6 of FIG. 5;

FIG. 7 is an example of a tip of an anchoring element of a prior art epicardial electrode; and

FIG. 8 is an enlargement of the tip of one of the anchoring elements of the epicardial electrode of FIG. 1, in accordance with the invention.

### DESCRIPTION OF THE PREFERRED EMBODIMENT

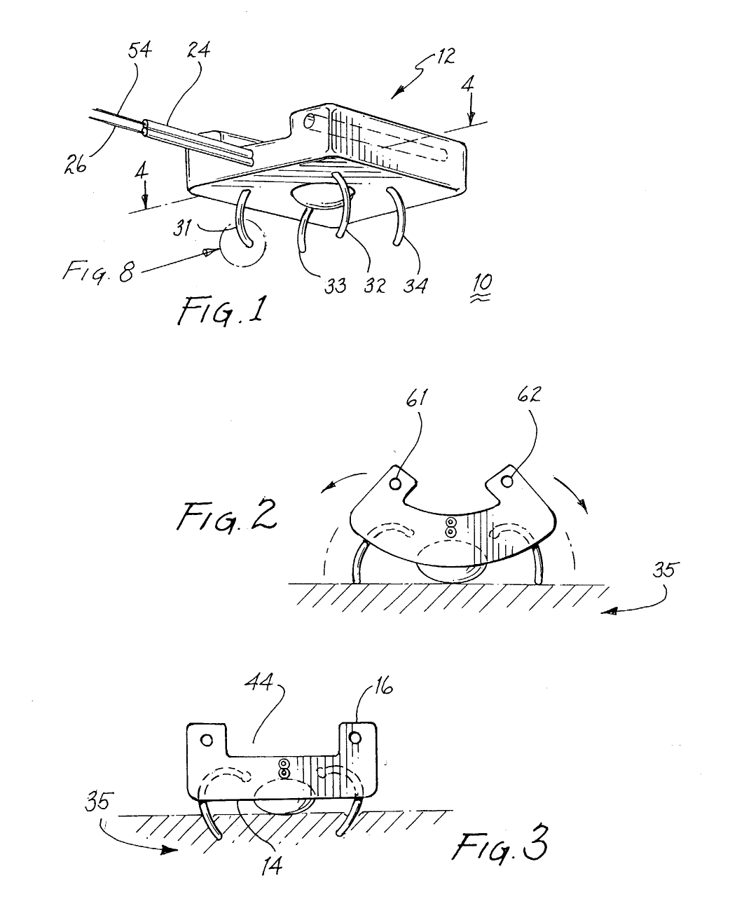

FIG. 1 is a perspective view of an epicardial electrode 10 in accordance with the invention. The epicardial electrode 10 comprises a generally parallelepiped flexible body 12 having a first side 14, a second side 16 opposite the first side, a lead side 18, and a back side 20 opposite the lead side. The flexible body 12 is preferably made from one of silicone rubber and polyurethane. The epicardial electrode 10 has a steroid eluting electrode element 22 attached to the first side 14 at the center of the first side for conveying electrical stimulation to cardiac muscle, and a lead 24 attached to the flexible body 12 at the lead side 18. The proximal end 25 of the lead 24 is electrically coupled to a pacemaker (not shown) that is implanted in the body. The portion of the electrode element 22 that protrudes from the flexible body 12 has the shape of a spherical segment. The lead 24 has at least an insulated cathode conductor 26 electrically coupled to the electrode element 22.

FIG. 2 shows a front view of the epicardial electrode 10 in a flexed position. The epicardial electrode 10 also comprises two pairs of prongs 31-34. Preferably, the prongs 31-34 are made from a NITINOL™ metal alloy; alternatively, they are made from another metal alloy. The prongs 31-34 protrude from the first side 14 of the flexible body 12. Each prong 31-34 has an external tip, or tip 41-44 at the end of the prong that is outside the flexible body 12 for penetrating the myocardium of the heart from the epicardium. The myocardium has many coronary vessels, and they are very susceptible to injury from sharp needles. Advantageously, the tip 41-44 of each prong 31-34 is dull so as not to cause excessive bleeding or trauma to the myocardium 35 (see FIG. 3). The prongs 31-34 advantageously traverse the myocardium 35 by passing between its muscle fibers and by pushing aside the capillaries within the myocardium. Whereas, the sharp-tip needles of the prior art are more likely to disadvantageously penetrate individual muscle fibers, thereby causing bleeding, and/or disadvantageously tear the capillaries of the myocardium, thereby causing more bleeding. The prongs 31-34 are electrically insulated from the electrode element 22. Each prong 31-34 is curved. The prongs 31-32 of the first pair of prongs are curved toward each other such that the prongs of the first pair are in a single plane approximately perpendicular to the first side 14. Similarly, the prongs 33-34 of the second pair of prongs are curved toward each other such that the prongs of the second pair are in another single plane approximately perpendicular to the first side 14.

FIG. 3 shows a front view of the epicardial electrode 10 in a relaxed position, such as after it has been deployed onto the heart. The flexible body 12 has a generally parallelepiped cavity 44 on the second side 16 to facilitate flexion of the flexible body. The cavity 44 extends from the lead side 18 to the back side 20. In an alternative embodiment (not shown), the flexible body 12 does not have any cavity on the second side 16, and the second side is substantially planar.

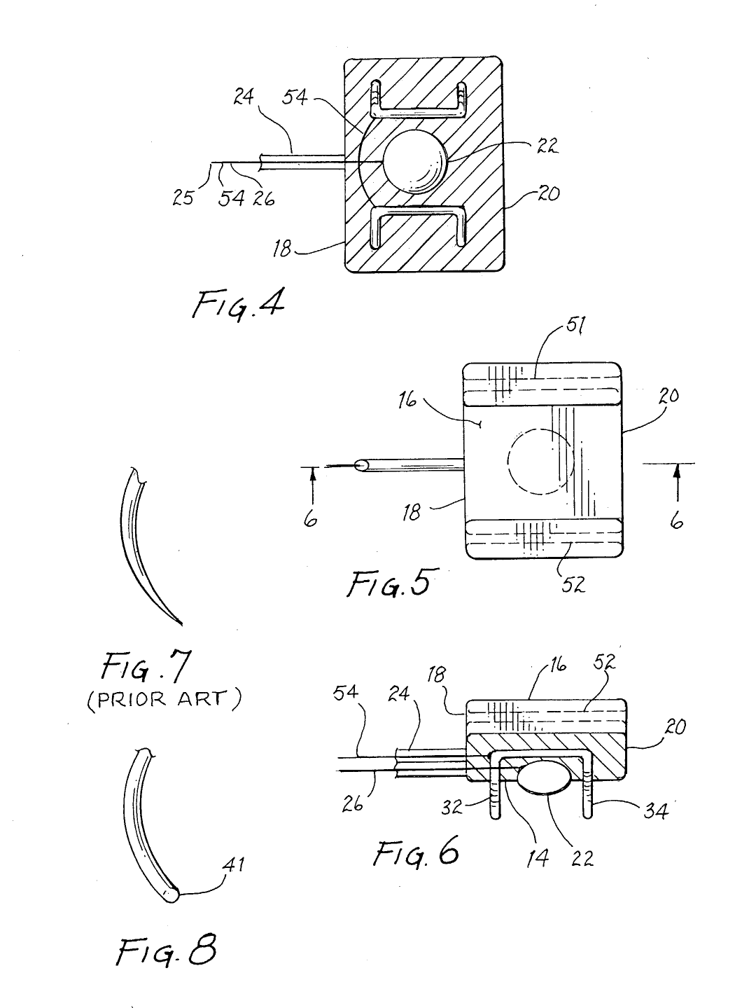

FIG. 4 is a cross-sectional view of the epicardial electrode 10 through cut-line 4-4 of FIG. 1. The lead 24 also has an insulated anode conductor 54 electrically coupled to one or more of the prongs 31-34, thereby producing a two-pole epicardial electrode 10. Alternatively, the lead 24 has only the insulated cathode conductor 26, thereby producing a single-pole epicardial electrode 10.

Referring now to FIG. 5, which shows a plan view of the epicardial electrode 10, and to FIG. 6, which shows a cross-sectional view of the epicardial electrode through cut-line 6-6 of FIG. 5. The flexible body 12 has two elongate holes 51-52 distal from the first side 14. The elongate holes 51-52 are on opposite sides of the parallelepiped cavity 44. The elongate holes 51-52 have an elongate axis parallel to the first side 14 and perpendicular to the lead side 18. The two elongate holes 51-52 are sized to accept rods of an applicator tool (not shown) through two openings 61-62 on the lead side 18 of the flexible body 12.

FIG. 7 shows the tip of an anchoring element of a typical prior art epicardial electrode, showing a disadvantageous, needle-like tip.

FIG. 8 is an enlargement of the tip 41 of prong 31 of the epicardial electrode 10 in accordance with the invention, showing a dull tip. The tips 42-44 of prongs 32-34 are similarly dull.

The epicardial electrode 10 is placed in operative position on the heart by applying force to flex the flexible body 12 such that the first side 14 becomes convex, as shown in FIG. 2. While flexed, the epicardial electrode 10 is positioned at a predetermined location on the heart such that the electrode element 22 is in intimate contact with the heart. While the electrode element 22 is in intimate contact with the heart, the force is removed to allow the first side 14 to return to a planar shape, causing the prongs 31-34 to penetrate the myocardium 35 atraumatically, thereby anchoring the epicardial electrode 10 to the heart, as shown in FIG. 3. Preferably, the prongs penetrate the myocardium of the ventricle about 0.5-1.0 mm. The epicardial electrode 10 in accordance with the invention is implanted manually during a sternotomy or a thoracotomy procedure, or using an applicator tool during an endoscopic, or minimally invasive, procedure.

By the term “dull” it is meant that the tip of each anchoring element is not sharp, pointed, barbed, fishhook-like, or needle-like; but rather, it is blunt, rounded, and smooth.

While the present invention has been described with respect to preferred embodiments thereof, such description is for illustrative purposes only, and is not to be construed as limiting the scope of the invention. Various modifications and changes may be made to the described embodiments by those skilled in the art without departing from the true spirit and scope of the invention as defined by the appended claims. For example, while the current design is intended for deploying on the ventricle, a smaller version could be used for deploying on the atrium.

I claim:

1. An epicardial electrode, comprising:
a generally parallelepiped flexible body having a first side, a back second side opposite the first side, a lead side, and a side opposite the lead side;
an electrode element attached to the first side at the center of the first side for conveying electrical stimulation to cardiac muscle;
a lead attached to the flexible body at the lead side, the lead having at least an insulated cathode conductor electrically coupled to the electrode element; and
two pairs of prongs insulated from the electrode element, each prong protruding from the first side of the flexible body, the external tip of each prong being dull, thereby allowing anchoring of the epicardial electrode to the cardiac muscle with minimal trauma to the cardiac muscle.

2. The epicardial electrode of claim 1, in which the prongs are curved and the prongs of a pair are curved toward each other such that the prongs of the pair are in a single plane approximately perpendicular to the first side.

3. The epicardial electrode of claim 1, in which the electrode element has the shape of a spherical segment.

4. The epicardial electrode of claim 3, in which the lead also has an insulated anode conductor electrically coupled to at least one of the prongs.

5. The epicardial electrode of claim 1, in which the flexible body has two elongate holes distal from the first side, each of the two elongate holes being on opposite sides of the flexible body, the elongate holes having an elongate axis parallel to the first side and perpendicular to the lead side.

6. The epicardial electrode of claim 5, in which the two elongate holes are sized to accept rods of an applicator tool through two openings on the lead side of the flexible body.

7. The epicardial electrode of claim 6, in which the electrode element has the shape of a spherical segment.

8. The epicardial electrode of claim 7, in which the lead also has an insulated anode conductor electrically coupled to at least one of the prongs.

9. The epicardial electrode of claim 1, in which the flexible body has a generally parallelepiped cavity on the second side, the cavity extending from the lead side to the side opposite the lead side, and in which the flexible body has two elongate holes distal from the first side, the elongate holes being on opposite sides of the parallelepiped cavity, the elongate holes having an elongate axis parallel to the first side and perpendicular to the lead side.

10. The epicardial electrode of claim 9, in which the two elongate holes are sized to accept rods of an applicator tool through two openings on the lead side of the flexible body.

11. The epicardial electrode of claim 10, in which the electrode element has the shape of a spherical segment.

12. The epicardial electrode of claim 11, in which the lead also has an insulated anode conductor electrically coupled to at least one of the prongs.

13. An epicardial electrode, comprising:
a generally parallelepiped flexible body having a first side, a back second side opposite the first side, a lead side, and a side opposite the lead side, and in which the flexible body has two elongate holes distal from the first side, each of the two elongate holes being on opposite sides of the flexible body, the elongate holes having an elongate axis parallel to the first side and perpendicular to the lead side;
an electrode element attached to the first side at the center of the first side for conveying electrical stimulation to cardiac muscle;
a lead attached to the flexible body at the lead side, the lead having at least an insulated cathode conductor electrically coupled to the electrode element; and
two pairs of prongs insulated from the electrode element, each prong protruding from the first side of the flexible body.

14. The epicardial electrode of claim 13, in which the two elongate holes are sized to accept rods of an applicator tool through two openings on the lead side of the flexible body.

15. The epicardial electrode of claim 14, in which each prong has a tip at the end of the prong outside the flexible body for penetrating the myocardium of the heart and in which each tip is dull to facilitate the prong to penetrate the myocardium atraumatically.

16. The epicardial electrode of claim 13, in which the flexible body has a generally parallelepiped cavity on the second side to facilitate flexion of the flexible body, the cavity extending from the lead side to the side opposite the lead side, and in which the two elongate holes are on opposite sides of the parallelepiped cavity.

17. The epicardial electrode of claim 16, in which the two elongate holes are sized to accept rods of an applicator tool through two openings on the lead side of the flexible body.

18. The epicardial electrode of claim 17, in which each prong has a tip at the end of the prong outside the flexible body for penetrating the myocardium of the heart and in which each tip is dull to facilitate the prong to penetrate the myocardium atraumatically.

19. A method of placing an epicardial electrode in operative position on a heart, the epicardial electrode having a flexible body with a planar first side, the first side having an electrode element attached thereto for stimulating cardiac muscle, and two pairs of prongs protruding from the first side for anchoring the epicardial electrode to myocardium, the tip of each prong being dull, comprising the steps of:
(a) applying force to flex the flexible body such that the first side becomes convex;
(b) while flexed, positioning the epicardial electrode at a predetermined location on the heart such that the electrode element is in intimate contact with the heart; and
(c) while the electrode element is in intimate contact with the heart, removing the force to allow the first side to return to a planar shape, causing the prongs to penetrate the myocardium, thereby anchoring the epicardial electrode to the heart.

20. The method of claim 19, in which the flexible body has two elongate holes sized to accept rods of an applicator tool, and which includes an additional step, prior to step (a), of inserting the rods of the applicator tool into the elongate holes, and in which the applying of force in step (a) and the removing of the force in step (c) are performed with the applicator tool.
